(19) United States
(12) Patent Application Publication
(10) Pub. No.: US 2023/0094165 A1
(43) Pub. Date: Mar. 30, 2023

(54) SENSOR MOUNTING FEATURES IN A CUSTOM-FITTED HEARING DEVICE SHELL

(71) Applicant: STARKEY LABORATORIES, INC., Eden Prairie, MN (US)

(72) Inventors: Justin Barlow, Minneapolis, MN (US); Kyle Olson, St. Louis Park, MN (US); Ganesh Borra, Minneapolis, MN (US); Peter Flanagan, Minneapolis, MN (US); Craig C. Feldsien, Prior Lake, MN (US); Michael Karl Sacha, Chanhassen, MN (US)

(21) Appl. No.: 17/939,417

(22) Filed: Sep. 7, 2022

Related U.S. Application Data
(60) Provisional application No. 63/250,373, filed on Sep. 30, 2021.

(51) Int. Cl. A61B 5/00; A61B 5/024

(52) U.S. Cl. CPC A61B 5/6817; A61B 5/02416; A61B 5/7207; A61B 5/746; A61B 2560/0406

(57) ABSTRACT

An ear-wearable electronic device has a shell with an outer surface. A mounting void goes through the outer surface of the shell and exposes an internal volume of the shell. The mounting void is located at an ear-contacting region of the shell. The ear-wearable device includes a photoplethysmography sensor assembly having an optical transmission structure mounted in the mounting void and having a distal end exposed proximate the outer surface. The distal end of the optical transmission structure conforms to the outer surface of the shell at the ear-contacting region. The distal end is in contact with ear tissue of a user of the ear-wearable electronic device during use.

### RELATED PATENT DOCUMENTS

This application claims the benefit of U.S. Provisional Application No. 63/250,373, filed on Sep. 30, 2021, which is incorporated herein by reference in its entirety.

### SUMMARY

This application relates generally to ear-level electronic systems and devices, including hearing aids, personal amplification devices, and hearables. For example, a custom-fitted, hearing device shell includes sensor mounting features that ensure good sensor placement in a custom fitted shell. In one embodiment, an ear-wearable electronic device includes a shell having an outer surface. A mounting void goes through the outer surface of the shell and exposes an internal volume of the shell. The mounting void is located at an ear-contacting region of the shell. The ear-wearable device includes a photoplethysmography sensor assembly having an optical transmission structure mounted in the mounting void and having a distal end exposed proximate the outer surface. The distal end of the optical transmission structure conforms to the outer surface of the shell at the ear-contacting region. The distal end is in contact with ear tissue of a user of the ear-wearable electronic device during use.

In another embodiment, an ear-wearable electronic device includes a shell having an outer surface. A mounting void goes through the outer surface of the shell and exposes an internal volume of the shell. The mounting void is located at an ear-contacting region of the shell. A biometric sensor assembly has a distal end exposed proximate the outer surface. The distal end of the biometric sensor conforms to the outer surface of the shell at the ear-contacting region. The distal end is in contact with ear tissue of a user during use. A compliant mounting structure is disposed between the biometric sensor and the mounting void. The compliant mounting structure causes a pressure applied between the distal end of the biometric sensor and the ear tissue to be within a predetermined pressure range during the use of the ear-wearable device.

The above summary is not intended to describe each disclosed embodiment or every implementation of the present disclosure. The figures and the detailed description below more particularly exemplify illustrative embodiments.

### BRIEF DESCRIPTION OF THE DRAWINGS

The discussion below makes reference to the following figures.

FIG. 1 is an illustration of a hearing device according to an example embodiment;

FIG. 2 is an illustration of ear geometry applicable to a hearing device;

FIGS. 3 and 4 are perspective views showing faceplate and cable features according to an example embodiment;

FIGS. 5, 6, and 7 are perspective views showing biometric sensor mounting features according to example embodiments;

FIG. 8 is a graph showing how sensor-to-ear pressure can affect biometric sensor signals;

FIG. 9 is a perspective view of an elastomer boot according to an example embodiment;

FIGS. 10 and 11 are cross-sectional views of a biometric sensor installed in a shell with an elastomer boot according to an example embodiment;

FIG. 12 is a perspective view of the surface of the shell and sensor of FIGS. 10 and 11;

FIGS. 13-15 are side views showing additional sensor mounts according to example embodiments; and

FIGS. 16a-16i are three-dimensional CAD renderings show additional details of an ear-wearable electronic device according to example embodiments.

The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

Embodiments disclosed herein are directed to an ear-worn or ear-level electronic hearing device. Such a device may include cochlear implants and bone conduction devices, without departing from the scope of this disclosure. The devices depicted in the figures are intended to demonstrate the subject matter, but not in a limited, exhaustive, or exclusive sense. Ear-worn electronic devices (also referred to herein as “hearing aids,” “hearing devices,” and “ear-wearable devices”), such as hearables (e.g., wearable earphones, ear monitors, and earbuds), hearing aids, hearing instruments, and hearing assistance devices, typically include an enclosure, such as a housing or shell, within which internal components are disposed.

Custom fitted hearing devices can result in ear-worn electronics with enhanced performance and comfort. A custom-fitted device may be formed, for example, by taking a mold of the user's ear and then using the mold to create a device that fits the exact contour of the user's ear. Technological developments such as three-dimensional (3D) scanning and 3D printing can increase the dimensional accuracy of custom-fitted device compared to, for example, molding of the part. Also, 3D scanning and 3D printing can increase the speed and ease with which the ear-wearable devices can be produced. This allows creating an organically shaped shell for the device that is custom fit to the individual's ear geometry to a high accuracy, e.g., within 0.1 mm.

One application of interest in ear-wearable technologies is the sensing of biometric data in the ear. Through direct contact with the surfaces of the outer ear, e.g., near the ear canal, sensors can accurately detect body temperature, pulse rate, and other metrics related to blood flow, such as blood oxygen level. This can be useful in hearing-aid devices, which are intended for long-term wear and so can unobtrusively gather health data over long-periods of time while at the same time performing its primary function of conditioning and amplifying sounds into the ear.

It has become increasingly cost-effective to perform in-ear sensing in ear-wearable devices due to the availability of low-cost yet accurate micro-sensors. An ear-wearable hearing aid will already have at least a microphone for sensing sound that is to be amplified. Other sensors may also be used in such, such as accelerometers, temperature sensors, etc., which can improve the accuracy of the sound reproduction via digital signal processing. Thus ear-wearable device architectures already include electronics (e.g., microprocessor, digital signal processors) capable of receiving and processing sensor data, and so these devices are amenable to adding biometric sensors, including biometric sensors that contact the skin within the ear.

One issue with using surface mounted sensors in the ear is that it can be difficult to position such sensors on a custom-fitted shell. If the device shell is of a standard shape, such as a tapered cylinder, it is possible to use a standard, interchangeable sensor on a whole class of devices. For example, if ten different sizes/configurations are desired, then ten different designs can be produced, in some cases automatically, e.g., using parametric computer-aided modeling. Further, it may be cost effective to use injection molding for producing those sets of shells, which is one of the cheapest methods for making a large number of devices out of plastics.

If a custom-fitted shell is desired, then the advantages of mass production manufacturing may not available. Generally, a production run for a custom-fit part could just be one or two, thus traditional production methods such as injection molding would be cost prohibitive. One way of implementing a custom fit earpiece is to use a custom-fitted cover that is fitted over the end of a standard shape shell. However, such an arrangement would not be ideal for surface-mounted sensors that contact the skin, as sensors would be mounted in the shell and not the cover, and thus could not achieve direct contact. Accordingly, a system for producing individually fitted ear-wearable devices is described below, such devices utilizing ear-canal sensors that are custom placed for each ear for which it is fitted. The system allows the design and production of custom-fitted ear-wearables that utilize interchangeable sensors placed at or near a surface of the device shell for direct contact measurements. The device shells can have other features that are also customize-fitted, such as cable retention features. Such devices can be produced at scale at reasonable cost.

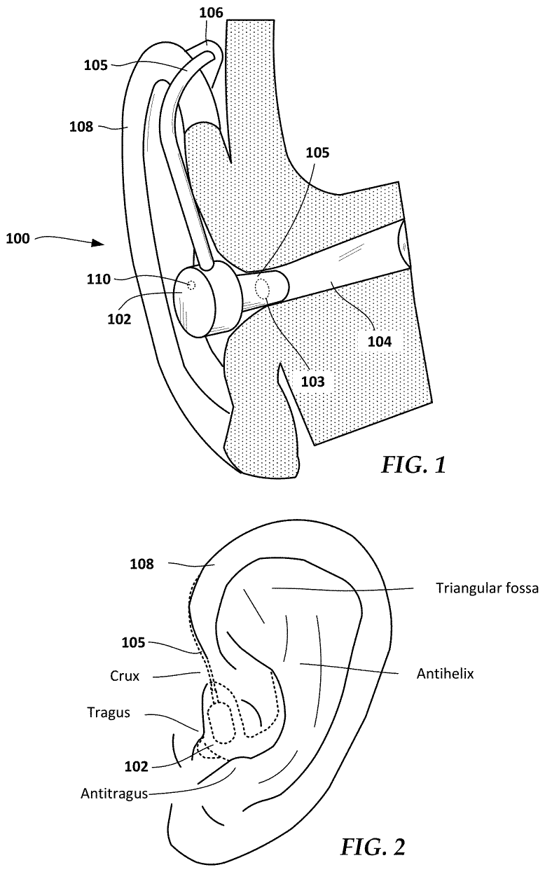

In FIG. 1, a diagram illustrates an example of an ear-wearable device 100 according to an example embodiment. The ear-wearable device 100 includes an in-ear portion 102 that fits into the ear canal 104 of a user/wearer. The ear-wearable device 100 may also include an external portion 106, e.g., worn over the back of the outer ear 108. The external portion 106 is electrically coupled to the internal portion 102. The in-ear portion 102 may include an acoustic transducer 103, where it is acoustically coupled to the ear canal 104, e.g., via a cable 105. The acoustic transducer 103 may be referred to herein as a “receiver,” “loudspeaker,” etc., however could include a bone conduction transducer. One or both portions 102, 106 may include an external microphone, as indicated by microphone 110. The configuration shown in FIG. 1 is referred to as receiver-in-canal (RIC), in that the receiver 103 is located in or proximate the ear canal 104, while other electronics are housed in the external portion 106, all being electrically coupled by the cable 105.

Other components of hearing device 100 not shown in the figure may include a processor (e.g., a digital signal processor or DSP), memory circuitry, power management and charging circuitry, one or more communication devices (e.g., one or more radios, a near-field magnetic induction (NFMI) device), one or more antennas, buttons and/or switches, for example. The hearing device 100 can incorporate a long-range communication device, such as a Bluetooth® transceiver or other type of radio frequency (RF) transceiver.

While FIG. 1 shows one example of an ear-wearable device, often referred to as a hearing aid (HA), the term hearing device of the present disclosure may refer to a wide variety of ear-level electronic devices that can aid a person with impaired hearing. This includes devices that can produce processed sound for persons with normal hearing. Some features described herein that are implemented in a RIC hearing device may also be used in other devices, such as behind-the-ear (BTE), in-the-ear (ITE), in-the-canal (ITC), invisible-in-canal (IIC), receiver-in-the-ear (RITE) or completely-in-the-canal (CIC) type hearing devices or some combination of the above. Throughout this disclosure, reference is made to a “hearing device” or “ear-wearable device,” which is understood to refer to a system comprising a single left ear device, a single right ear device, or a combination of a left ear device and a right ear device.

In existing RIC designs, the in-ear portion 102 can be quite small, just housing the receiver 103 and possibly the microphone 110, while all other electronics are located in the external portion 106. Although the external portion 106 could include biometric sensors, the in-ear portion 102 is the best place to incorporate these sensors. The external portion 106 may still be needed, as it would be less than ideal to locate all the electronics and power supply in a custom, in-ear, shell. Thus, the designs described herein include an external portion 106 (also referred to as a RIC body) with a custom fitted in-ear portion (also referred to as sensor shell). The combination of the external portion 106 and a custom shell in-ear portion 102 can be used to produce a hearing device with health/biometric sensors.

As noted above, one challenge in making custom fitted ear-wearable devices that can be produced at scale involve integrating sensors into the complex, organically shaped outer shell that is unique for each ear. Another challenge is aligning other components with the ear, such as cables that extend from the devices. In FIG. 2, a diagram of the outer ear 108 shows a part of the cable 105 extending along the crux of the ear (also referred to as the external auditory meatus). The location and dimensions of the crux will differ slightly for every ear, but for optimum comfort and fit, the cable 105 should be aligned with the crux within a few degrees as it exits the in-ear portion 102 (seen in FIG. 2).

The shell will incorporate mounting features that secure the cable to the shell and provide the cable with the desired alignment. Because part of the shell will be visible in the user's ear, it is desirable to improve the aesthetic of the adhesive system for the cable while maintaining the desired robustness and reliability of the hearing device. In embodiments described below, this involves a specially designed cable exoskeleton and a shell-integrated cable retention that adheres the cable to the sensor shell.

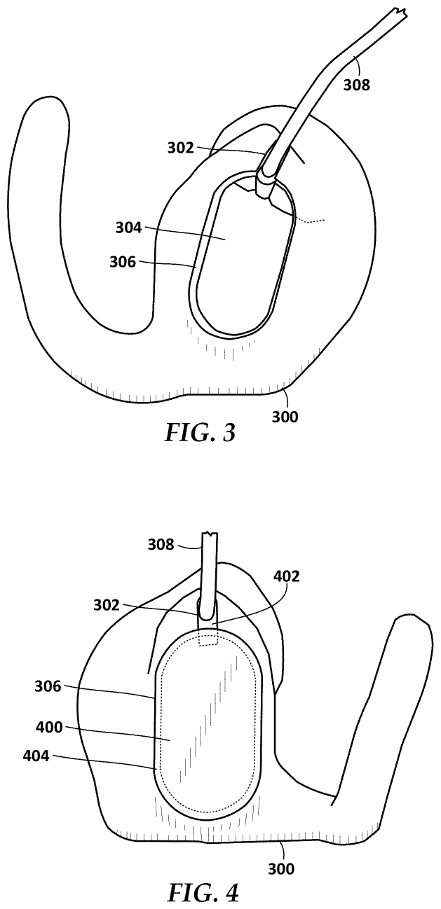

In FIG. 3, a perspective view shows cable retention features of an ear-wearable device according to an example embodiment. The ear-wearable electronic device includes a shell 300 with a cable retention slot 302. The shell 300 also optionally includes a faceplate void 304 that has a curved and beveled perimeter edge 306. The faceplate void 304 facilitates access to one or more devices installable into the shell 300. The cable retention slot 302 intersects the beveled perimeter edge 306, such that a cable 308 coupled to internal electronics (e.g., sensors, not shown) at a distal end can be introduced into the faceplate void 304. After the internal electronics are fastened to their respective mounting structures, the cable 308 can be secured into the cable retention slot 302. The cable 308 is coupled to electronics inside the shell 300 that will be discussed in detail below.

The shell 300 can be 3D printed using a liquid resin process that utilizes a resin for audiology applications, such as provided by pro3dure® medical LLC, e.g., GR-1 resin. This resin may also be used for skim coating as described elsewhere herein. Various adhesives may be used to secure devices to the shell, such as rigid adhesives (e.g., Loctite® 4307) and silicone adhesives (e.g., Loctite® 5056). The shell 300 may be oriented during printing such that the faceplate void 304 is aligned with (e.g., facing) the build platform, with the canal tip being the last scaffolding printed. This ensures that the more critical tolerances (e.g., those that can adversely affect fit in the ear) are formed in the X-Y dimensions and not in the Z-dimension, which is not as controllable due to the Z-dimension, which is controlled by the thickness of resin that is hardened by ultraviolet light for each layer. For example, Z-direction tolerance may be as high as 0.012″ worst case, which is higher than the worst case tolerances in the X-Y directions.

In FIG. 4, the shell 300 is shown with a faceplate 400 with a beveled edge 404 that mates with the perimeter edge 306. The faceplate 400 has an unbroken covering surface that matches the outer surface of the shell 300 surrounding the faceplate void 304 and traps the cable 308 into the cable retention slot 302. A biocompatible filler 402 (e.g., silicone or rigid adhesive) can be backfilled into the cable retention slot 302 after the faceplate 400 is installed, which creates a gasket around the cable 308 at its exit point from the shell 300. The filler 402 seals off the shell 300 and acts as a strain relief. Using a material such as silicone (e.g., Loctite® 5056) for the filler 402 provides an aesthetically pleasing appearance even after the expected life-cycle of wear and tear on the ear-wearable device.

The cable 308 includes conductors that electrically couple an external controller (e.g., external portion 106 shown in FIG. 1A) with a receiver (e.g., receiver 103 shown in FIG. 1A) mounted within the shell 300. The shell 300 also includes one or more sensors that are also coupled to the controller by conductors of the cable 308. In particular, a sensor near the surface of the shell 300 is provided that is in close proximity to an ear surface enabling accurate biometric measurements to be made. As noted above, it can be challenging to accurately place such sensors in a custom-fitted shell, as well as providing for straightforward assembly of the sensor into the shell. In embodiments described below, mounting features and assembly methods are described for a near-surface-mounted sensor, such as a photoplethysmography (PPG) sensor.

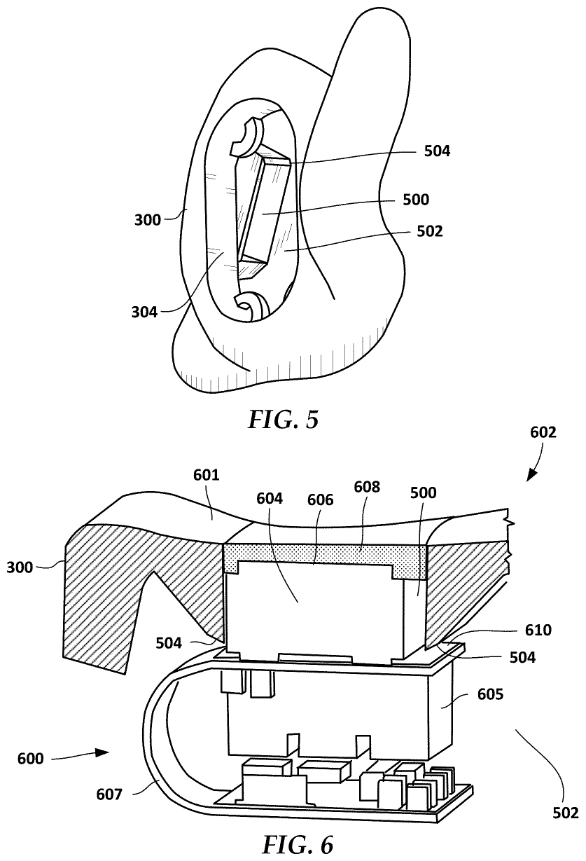

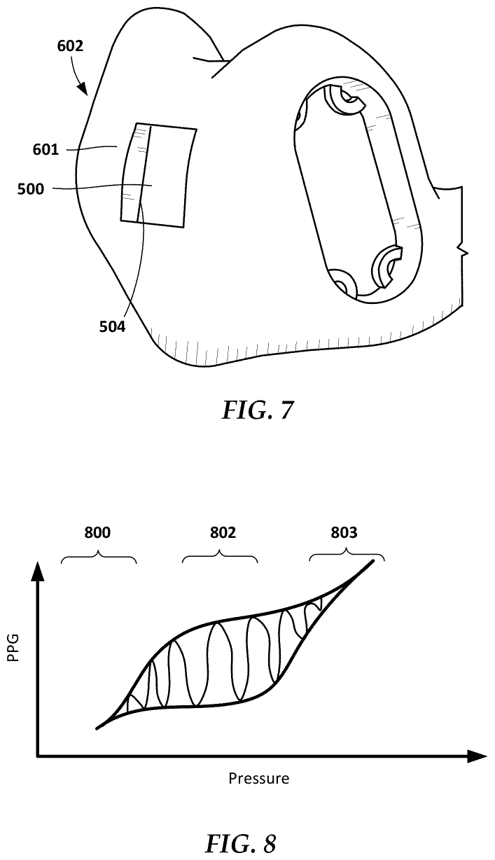

In FIGS. 5-7, perspective views of the shell 300 show details of PPG sensing mounting features according to an example embodiment. As seen in FIG. 5, a mounting void 500 through the outer surface of the shell 300 exposes an internal volume 502 of the shell 300. As best seen in FIGS. 6 and 7, the mounting void 500 is located at an ear-contacting region 602 of the shell 300, which is placed near to or in contact with an ear surface, e.g., a tragal wall of the tragus or antitragus, during use. Other ear locations, such as in or near the ear-canal can also be used. The geometry of the shell 300 near the ear-contacting region 602 may deviate from the ear geometry (e.g., be enlarged compared to the ear mold) in some embodiments causing an interference fit between the outer surface 601 of the shell near the PPG sensor and a surface of the user's ear.

In FIG. 6, a PPG sensor assembly 600 is shown installed in the mounting void 500. The PPG sensor assembly 600 has an optical transmission structure 604 mounted in the mounting void 500. The optical transmission structure 604 may include a lens, waveguide, and or other optical transmitting and shaping components, e.g., polarizers, filters, etc. The optical transmission structure 604 has a distal end 606 exposed proximate the outer surface 601. A skim coating 608 is applied over the distal end 606 of the optical transmission structure 604. The skim coating 608 conforms to the outer surface 601 of the shell 300 at the ear-contacting region 602, and is in contact with ear tissue of the user during use. Generally, the skim coating 608 is a material (e.g., resin) that will adhere to the shell material, and also has favorable optical characteristics (e.g., transparency, index of refraction) at the wavelengths used by the PPG sensor assembly 600.

The PPG sensor assembly 600 also has an opto-electronics section 605 that includes optical transducers (e.g., photodetectors, light emitting diodes), signal conditioning circuit components (e.g., amplifiers, filters), power conditioning circuits, etc. The size of the entire PPG sensor assembly 600 is such that it can pass through the faceplate void 304. Some parts of the PPG sensor assembly 600 may be flexible, such as flex cable 607, which can make the PPG sensor assembly 600 fit more compactly inside of the shell 300. Other circuitry may be installed on the flex cable 607 that are unrelated to PPG sensing, such as power regulators, other sensors, etc.

A shoulder 504 is formed contiguously with the shell 300 and extends into the internal volume 502 of the shell. The shoulder 504 is positioned relative to the outer surface 601 of the shell 300 such that the distal end 606 of the optical transmission structure 604 is proximate to the outer surface 601. Generally, close proximity of the optical transmission structure 604 to the ear tissue ensures an accurate PPG reading. As seen in FIG. 6, the PPG sensor assembly 600 has a mating surface 610 that fits up against the shoulder 504. Contact between the mating surface 610 and shoulder 504 provides accurate depth control for sensor mounting. The shell 300 may include retaining features such as clips or snaps for at least temporarily holding the PPG sensor assembly 600 in place, and an adhesive (e.g., rigid adhesive) can be used for permanent mounting.

A PPG sensor as described above can be used in an ear-wearable device to allow the users to track biometric data such as heart rate and oxygen saturation level (SpO2). As noted above, the ear-contacting region of the shell may be enlarged or have other feature that ensure that the PPG sensor assembly (or at least an optical transmitting structure coupled to the sensor) is pressed against the ear tissue to ensure accurate readings. The amount of pressure between the sensor and the ear should fall is a certain range to balance user comfort with PPG sensing performance, as well as improving the manufacturability of the PPG sensor assembly process.

Health sensors are often placed as close to the skin as possible to obtain the most accurate readings. In a custom fitted hearing device, the sensor placement should provide repeatable and reliable operation, while not causing discomfort to the user during long-term wear. To do this, supporting features, such as pockets, brackets, bridges, etc., as described above, can be used to ensure the sensor maintains a consistent placement and depth with respect to shell outer surface. There is also an ideal pressure range where the circulation visibility is optimal for PPG sensor to capture a strong signal for data acquisition. When the pressure exerted by the PPG sensor is too high, circulation could be blocked, which can reduce or eliminate the sensed phenomena. When the PPG sensor is not exerting enough pressure, the visibility can be obstructed by an increased gap between the sensor and the blood circulation region.

In FIG. 8, a graph shows a general relationship between PPG accuracy and sensor-to-ear pressure according to an example embodiment. In region 800, the pressure is too low, which results in low signal amplitude (here shown as the difference between highest and lowest peak of the signal) due to poor sensor visibility. In region 803, sensor visibility is good, but the blood circulation is locally restricted, again resulting in a low signal amplitude. The PPG sensor and shell should be fitted to strike an ideal normal pressure as shown in region 802. This fitting can be obtained via geometric design and by material selection. In one embodiment, sensor pressure (e.g., average pressure applied between the ear and the sensor by flexible supports) should be below arterial pressure (˜<32 mm Hg). Note that the relationship between pressure and force is well known, e.g., pressure=force/area. Therefore, the terms pressure and force may be used interchangeably herein. The applied area is typically known (or is easy to measure or estimate) and can therefore readily enable converting between force and pressure.

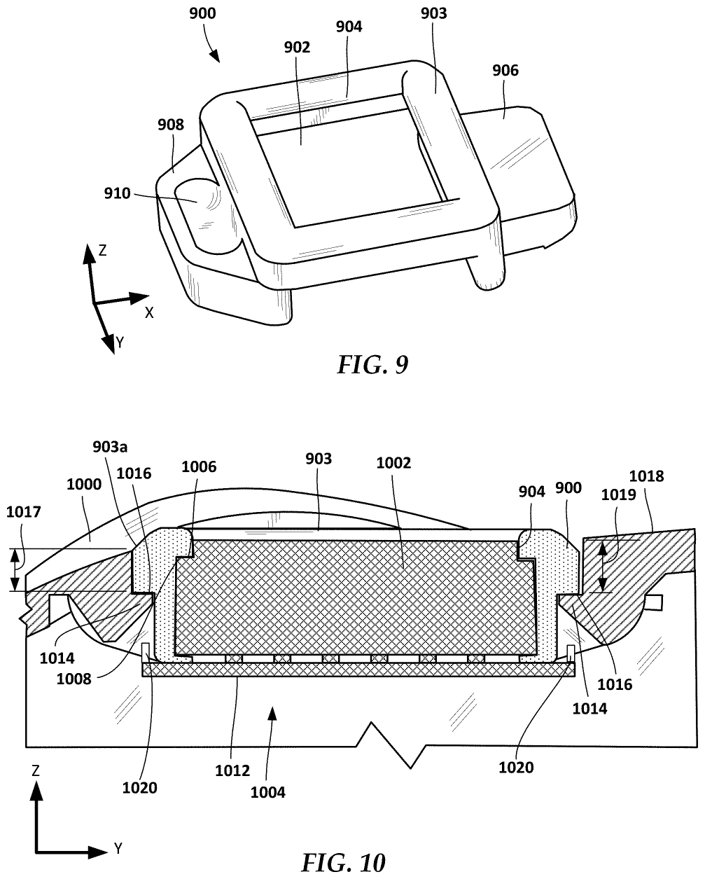

In one embodiment, a PPG sensor as described above can be supported in the shell by an elastomer boot. In FIG. 9, a perspective view shows an elastomer boot 900 according to an example embodiment. The elastomer boot 900 includes a mounting hole 902 with an internal perimeter 904 that is part of a gasket 903 that surrounds an outward facing part of the sensor assembly. Part of the sensor assembly (e.g., similar to the optical transmission structure 604 in FIG. 6) is pressed through the hole 902 of the elastomer boot 900 with light force until the sensor assembly cannot be pressed any further. The elastomer boot 900 features a snap finger 906 and a glue pocket wing 908 with a glue pocket 910, the function of which will be described in further detail below.

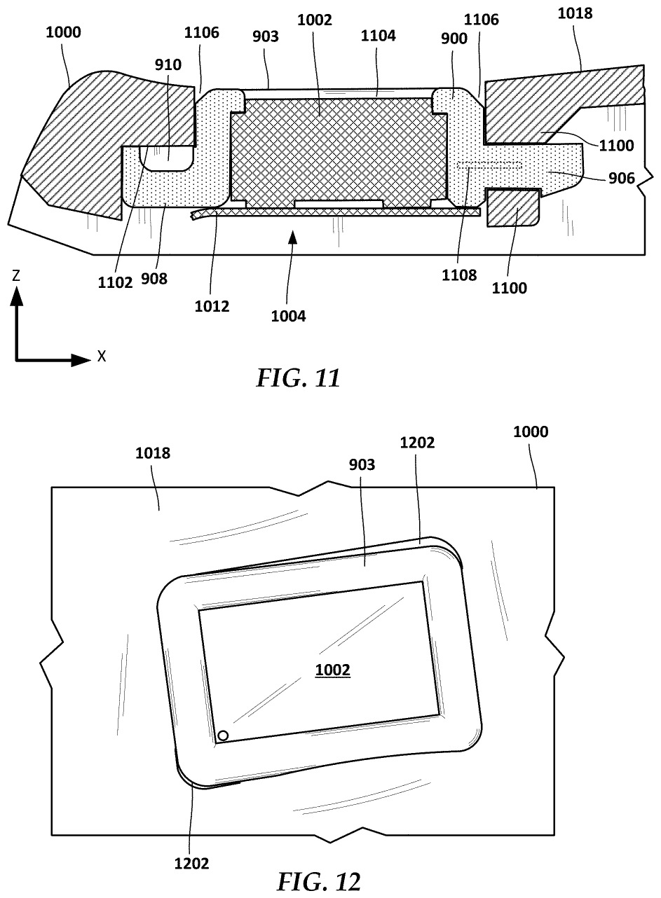

In FIGS. 10 and 11, cross sectional views show how the elastomer boot 900 interfaces with a shell 1000 according to an example embodiment. This shell 1000 may include other features of the previously illustrated shell 300, but has different mounting features for the elastomer boot 900. As seen in FIG. 10, the elastomer boot 900 surrounds an optical transmission structure 1002 of a PPG sensor assembly 1004. The boot 900 has a stop surface 1006 against which a notch 1008 of the optical transmission structure 1002 rests. Both the stop surface 1006 and the notch 1008 extend partially or completely around the periphery of the mounting hole 902 and optical transmission structure 1002, respectively.

A The shell 1000 includes rigid snap fingers 1014 that the elastomer boot 900 can squeeze past during install. The rigid snap fingers 1014 have a shelf surface 1016 that the elastomer boot 900 sits against after installation. The distance between the shelf surface 1016 and an outer surface 1018 of the shell 1000 (e.g., a reference point near the ear-contacting region) will define how much the PPG sensor assembly 1004 will protrude from the shell 1000. Because of the shape of the outer surface 1018, this distance will vary around the perimeter of the PPG sensor assembly 1004 (see, e.g., distances 1017 and 1019), and so an average value of protrusion may be used to position the shelf surface 1016 in the shell 1000. The elasticity of the boot 900 will allow the PPG sensor assembly 1004 to deflect inward when the shell 1000 is placed in the user's ear.

As seen in FIG. 11, when the elastomer boot 900 and sensor assembly 1004 is installed, the snap finger 906 is inserted through a snap window feature 1100 formed in the shell 1000 which to locks the assembly's horizontal position (corresponding to the x-direction in the figure). With the glue pocket wing 908 accessible from the faceplate opening (e.g., faceplate void 304 in FIG. 3), the assembler can dispense a small amount of adhesive (e.g., Loctite™ 4307 UV-cured CA adhesive) into the pocket 910. After the adhesive is dispensed, the assembler presses the elastomer boot 900 through the shell's sensor wall utilizing the snap finger 906 as a lever until the gasket 903 is visibly fully sealed at the outer surface 1018 of the shell. The glue pocket wing 908 would be pressed against the shell interior surface 1102 and cured to fully assemble the PPG sensor assembly in place. A clear and transparent adhesive may be precisely dispensed above the sensor's glass lens in region 1104 to bond the elastomer boot 900 and the PPG sensor assembly together. Another option is two-shot injection mold where the gasket 903 and clear lens are of similar material. This could improve retention of the PPG sensor assembly and improve the robustness of the device.

A small amount of adhesive may optionally be flowed around the outer perimeter of the gasket in region 1106 to secure the outer perimeter to the wall of the shell 1000 and/or smooth out the interface between the gasket 903 and the outer surface 1018 of the shell 1000. Such adhesion could stiffen the elastomer boot 900 in the z-direction (which is normal to the ear tissue) and so may only be used if the elastomer boot 900 attached this way can sufficiently deflect to maintain the desired pressure during use of the ear-wearable device. Another possible approach to smooth the interface between the gasket 903 and the outer surface 1018 of the shell 1000 is to have shell modeling software smoothen out the shell so the region 1106 is levelled and/or angled to meet the chamfer features 903a (see FIG. 10) of the gasket 903.

In FIG. 12, a perspective view shows the outward facing surface of the optical transmission structure 1002 after installation of the PPG sensor assembly 1004 into the shell 1000. The gasket 903 surrounds the optical transmission structure 1002 and seals it within the shell 1000. Note the gaps 1202 seen near some edges of the gasket 903. These are due to some portions of the gasket 903 being above or below the outer surface 1018 of the shell 1000. This is expected due to the organic shape of the outer surface 1018. The gaps 1202 could be left as is, or be filled with a soft or rigid material to smooth the assembly and prevent the accumulation of dirt in the gaps 1202.

Because the outer surface of the shell 1000 is custom designed to fit a particular user's ear, the protrusion of the gasket 903 and optical transmission structure 1002 will vary for each device. Ideally, the amount of pressure (e.g., an average pressure over the exposed area of the sensor) applied between the sensor and ear should be within a predefined range even though the final geometry of each assembly will be unique. The CAD tools used to accurately place the internal features of the shell 1000 (e.g., interior surface 1102, snap window feature 1100, snap fingers 1014) can also calculate how much of the gasket 903 and optical transmission structure 1002 protrude. This can be used to estimate how forces will be applied to the PPG sensor assembly 1004 and whether the reactionary force provided by the boot 900 will result in the target pressure being applied during use. For example, a maximum protrusion for any part of the gasket 903 could be used to estimate final placement of the sensor assembly mounting features, and this could be adjusted based on other factors, e.g., whether there are multiple points near the maximum protrusion, location of the maximum protrusion relative to the optical transmission structure 1002 (e.g., side, corner), etc.

The elastomer boot 900 may be formed of any biocompatible elastomer with the desired modulus of elasticity, such as silicone. It is known that the softer silicone material can attract lint and messy residues (such as dirt) in general. There are specialty silicone processes that smoothen the surface to reduce the ingress adhesion. Other materials that may be used for the elastomer boot 900 may include, but are not limited to, silopren, ethylene propylene (EPDM), Viton, natural rubber, etc.

The pressure exerted on the user's ear by the PPG sensor assembly using an elastomer boot 900 is a function of at least the following parameters: the amount of protrusion of the PPG sensor assembly outside the shell, enlarged dimensions (if any) of the shell's surface proximate the PPG sensor assembly that locally tighten the fit, the modulus of elasticity of the boot material, the areas and type of attachment of the boot to the shell, and the geometry of the boot. All of these can be controlled in the design, and fine tuning of the assembly can be done to adjust the pressure, e.g., during an initial fit. For example, as noted above, some amount of adhesive may be applied around the periphery of the gasket resulting adhesion of the outer gasket walls to the shell. This could be used to tune the applied pressure of the sensor by increasing shear forces between the boot and the shell. In other embodiments, a boot or gasket of several different durometers may be made available during fitting available. An initial fit can provide enough data on the protrusion and pressure applied to fine tune the elastomer material that fits the user case. For instance, a tragus wall could be approximately flat, which means higher durometer can be used as the force is evenly distributed. Whereas the tragus wall that are curvy may require lower durometer to allow the PPG sensor to conform to the ear geometry more easily. Thus, in some embodiments not only and average pressure is considered, but a peak pressure may also be considered to ensure comfort when the device is worn.

The PPG sensor support structure (e.g., the elastomer boot 900) is designed to control the pressure exerted on the ear tissue, increasing measurement accuracy while at the same time maintaining comfort. Other biometric sensors may benefit from a pressure fit, such as thermal sensors, electrodes, etc. In order to more accurately determine the local pressure, a thin pressure sensor may be deployed in a mechanical coupling region between the shell and the biometric sensor. For purposes of this disclosure, a pressure sensor may include any sensor that provides a signal that can be converted to a local or average pressure exerted by the PPG sensor assembly against the ear. For example, sensors that detect force and/or displacement can be indicative of pressure using the appropriate conversion factors.

The pressure sensor can be used to assist when fitting the device, e.g., allowing for fine tuning of the sensor structure. The pressure sensor can also be used to alert the user when the pressure is too low, which could signify that the product is inserted in the ear incorrectly. Such a sensor could have other uses, such as generating a compensatory signal that could reduce motion artifacts by subtracting the pressure signal from the PPG signal where the motion artifact occurs. The pressure sensor signals could be used by other audio processing functions, e.g., feedback suppression, own voice suppression, occlusion detection, etc. All of these different functions could be used together on the same device.

In reference again to FIG. 10, a pressure sensor 1020 is shown according to an example embodiment. In this case, the pressure sensor would detect the effects of compression and expansion forces in the z-direction, e.g., by measuring a stretching of the sensor 1020. Such a pressure sensor 1020 could be incorporated directly into the PPG sensor assembly 1004. In this example, one end of the pressure sensor 1020 is affixed to a flex circuit/printed circuit board 1012, and an opposite end is affixed to the interior surface of the shell 1000. In other embodiments, two sensors (e.g., one on the flex circuit 1012 and one on the interior of the shell 1000) could be used to detect changes in distance from each other, which would correspond to a deflection that could be converted to pressure based on an elastic modulus of the boot and supporting structure. A single optical distance sensor could also be used, e.g., a time of flight sensor that measures the distance between a location on the flex circuit 1012 and a reflective surface inside the shell.

In some embodiments, the elastomer boot 900 itself could provide force/pressure indicators. For example, a conductive material could be embedded in the boot, e.g., evenly distributed throughout and/or printed as structures on a surface of the boot. The electrical characteristics (e.g., resistance, capacitance) of the conductive material may change sufficiently in response to boot compression/expansion to provide a pressure signal. The mechanical interface between the boot 900 and the sensor assembly 1004 could include an electrical interface (e.g., surface mounted conductive pads) for electrical connections therebetween.

In reference again to FIG. 11, region 1108 shows another possible location for a force/pressure sensor according to an example embodiment. This region 1108 would experience bending in response to forces applied to the PPG sensor assembly 1004 relative to the shell 1000. The location of the region 1108 could be selected to maximize bending of any sensors formed in or added to region 1108. Electrical couplings could be provided as described above, e.g., surface mounted conductive pads on both the boot 900 and sensor assembly 1004 that are in contact once the boot is installed.

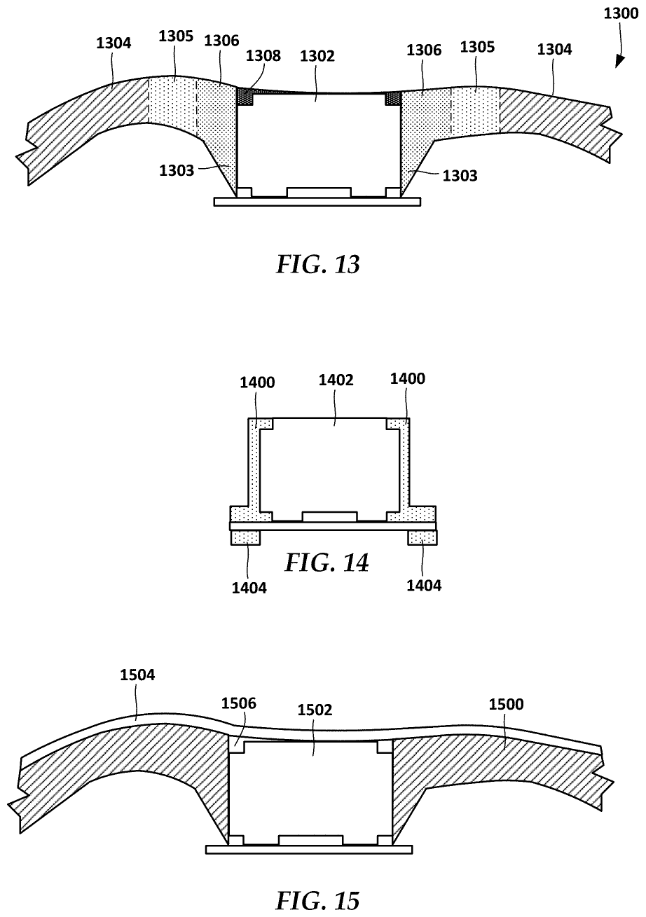

An elastomeric boot 900 as described above can provide a pressure-controlling mounting structure for a biometric sensor, and has features to assist in easy and accurate replacement. Although the illustrated internal structures of the shell 1000 with which the boot 900 interfaces (e.g., interior surface 1102, snap window feature 1100, snap fingers 1014) may take slightly more interior shell space than other designs (e.g., mounting shoulder 504 shown in FIGS. 5 and 6), this may be an acceptable tradeoff for the functional benefits provided by a flexible mount. Other embodiments of flexible or compliant mounts are shown in FIGS. 13-15.

In FIG. 13, a cross-sectional view shows a biometric sensor 1302 (e.g., PPG sensor assembly) mounted in a shell 1300. The shell 1300 includes mounting shoulders 1303 similar to other embodiments described above. In this case, the mounting region of the shell 1300 surrounding the biometric sensor 1302 has heterogeneous mechanical properties that are designed to provide a flexible mount for the biometric sensor 1302 and apply a predetermined pressure to the user's ear when the device is worn. In this example, two regions 1305, 1306 exhibit different material or structural properties (e.g., properties measured as a function of volume) that are different than another region 1304 of the shell 1300. The remainder of the shell 1300 may have the same material or structural properties as region 1304, which is the default property for the shell print materials.

Regions 1305 and 1306 may achieve different material or structural properties by different material compositions (e.g., different materials, different proportion of additives), different microstructures (e.g., voids, channels, fill percentage), different processing parameters (e.g., curing time, temperature), etc. While regions 1305 and 1306 are shown as homogenous regions, the material or structural properties could gradually change from the sensor mounting region outward according to a linear or non-linear gradient function. For example, modulus of elasticity of the shell material could linearly decrease from the outside of region 1305 (facing away from the biometric sensor 1302) towards the edge of region 1306 that is adjacent the biometric sensor 1302.

Generally, region 1305 may be more flexible than at least region 1304, assuming the shell 1300 is designed to be rigid in use. Region 1306 may be stiffer or more flexible than region 1305 in some embodiments. For example, it may be desirable to have higher stiffness at region 1306 to ensure good adhesion between the shell 1300 and the sensor 1302. Because region 1305 would experience bending as a primary mode of deflection in response to a force applied to the sensor 1302, it may provide sufficient flexibility. Also, small local deflections in region 1305 can result in relatively large deflections at the sensor 1302, thus reducing the amount of mechanical stress in the shell structure during deflection. Note that a skim coating 1308 may optionally be used in this embodiment.

In FIG. 14, a side view shows a biometric sensor 1402 (e.g., a PPG sensor assembly) according to another example embodiment. This biometric sensor 1402 can be used with any shell mounting features or intermediary structures (e.g. a boot) described above. The biometric sensor 1402 is encapsulated with a flexible coating 1400, e.g., a foam, elastomer, rubber, etc. The flexible coating 1400 can be applied by spraying, dipping, 3-D printing, etc. The flexible coating 1400 serves a similar function as the elastomer boot described above. The flexible coating 1400 can still provide a compliant mounting structure without adding additional mechanical retaining structures to the shell. For example the flexible coating can be fastened directly to the shell surfaces using an adhesive. If the shell has mechanical retaining features (e.g., similar to snap window feature 1100 shown in FIG. 11), then additional parts of the biometric sensor 1402 may be covered with a flexible coating material to ensure compliant mounting. The illustrated lower pads 1404 can provide a compliant interface for the lower surface of the biometric sensor 1402 in such an arrangement.

In FIG. 15, a cross-sectional view shows a biometric sensor 1502 (e.g., a PPG sensor assembly) in a shell 1500 according to another example embodiment. The shell 1500 and biometric sensor 1502 may use any mounting arrangement previously described, including an elastomeric boot, flexible encapsulation, direct mount, etc. A flexible conformal covering 1504 covers both the shell 1500 and outward facing end of the biometric sensor 1502. If the biometric sensor 1502 utilizes optical transmissions between the ear-wearable device and the ear tissue, then the conformable covering 1504 should be translucent or transparent around the wavelength of interest. For other types of biometric sensors, the properties of the conformal covering 1504 can be selected appropriately (e.g., electrical conductivity, heat conductivity, etc.).

The conformal covering 1504 can compress when the shell 1500 is inserted in the user's ear to control pressure between the ear and the sensor 1502, e.g., to maintain pressure within a desired range. The conformal covering 1504 may be applied adhesively to the shell's outer surface via spraying, dipping, 3D printing, or may be a cover made of fabric, elastomers, etc., that is slid over the outside surface of the shell 1500. In the latter case, the fitting of the conformal covering 1504 to the shell 1500 may be sufficient to hold the conformal covering 1504 in place, or an adhesive may be used tack at least some part of the conformal covering in 1504 plate. The conformal covering 1504 may be over the entire shell 1500, or over just regions surrounding the sensor 1502. The spaces 1506 between the shell 1500 and sensor 1502 may be filled (e.g., with a resin) before applying or installing the conformal covering 1504, or the spaces may be unfilled. Note that any of the embodiments shown in FIG. 10-15 may be used in combination (e.g., rubber boot as in FIG. 10 and/or a foam encapsulation as in FIG. 14 in combination with flexible shell as in FIG. 13). Also, a pressure sensor as shown in FIGS. 10 and 11 may be incorporated with flexible mounts as shown in FIGS. 13-15.

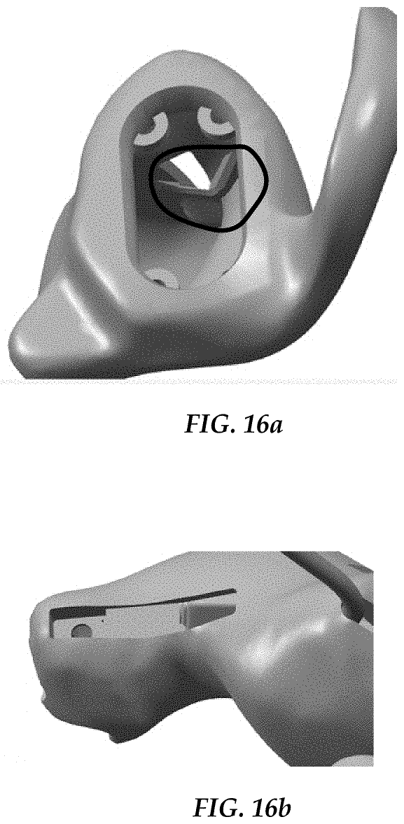

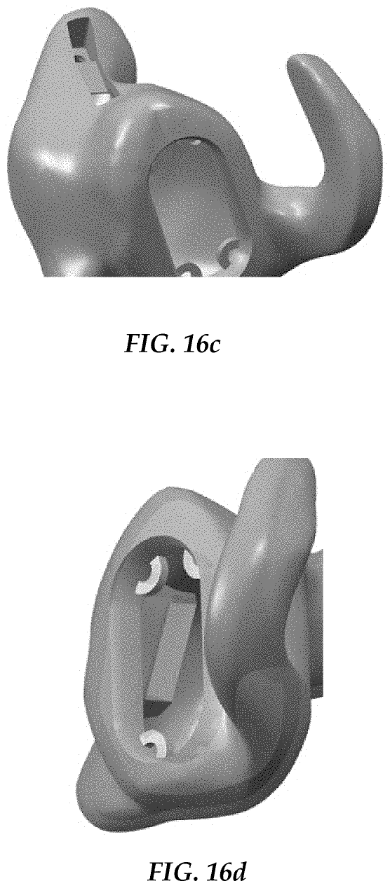

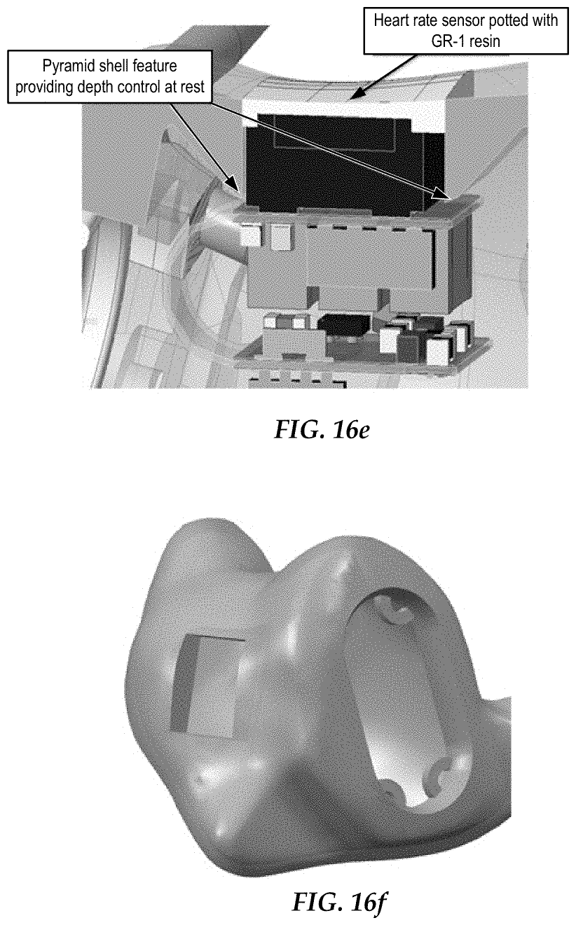

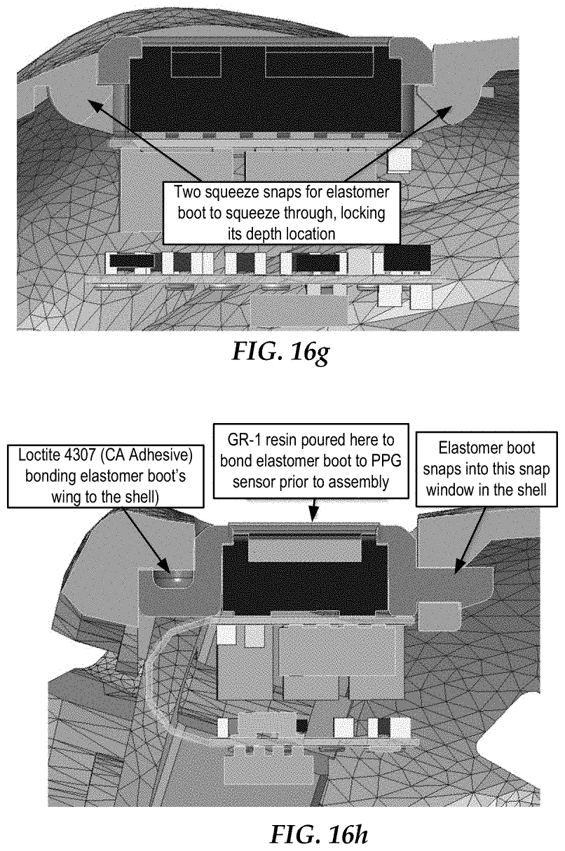

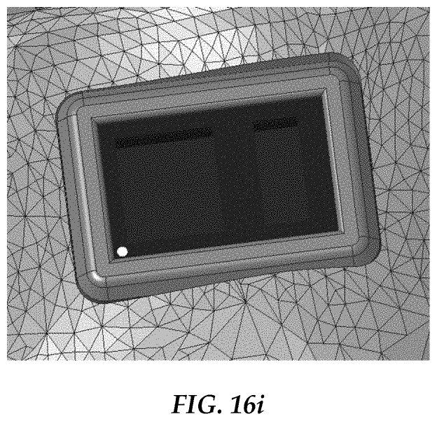

In FIGS. 16a-16i, three-dimensional CAD renderings show additional details of an ear-wearable electronic device according to example embodiments. This document discloses numerous example embodiments, including but not limited to the following:

Example 1 is an ear-wearable electronic device comprising: a shell having an outer surface; a mounting void through the outer surface of the shell that exposes an internal volume of the shell, the mounting void located at an ear-contacting region of the shell; and a photoplethysmography sensor assembly having an optical transmission structure mounted in the mounting void and having a distal end exposed proximate the outer surface, the distal end of the optical transmission structure conforming to the outer surface of the shell at the ear-contacting region, the distal end in contact with ear tissue of a user of the ear-wearable electronic device during use.

Example 2 includes the ear-wearable device of example 1, wherein the outer surface of the shell corresponds uniquely to an ear geometry of the user. Example 3 includes the ear-wearable device of examples 1 or 2, wherein the shell further comprises a shoulder formed contiguously with the shell extending into the internal volume of the shell, the shoulder positioned relative to the outer surface of the shell such that the distal end of the optical transmission structure is proximate to the outer surface. Example 4 includes the ear-wearable device of example 3, further wherein the distal end of the optical transmission structure comprises a skim coating that conforms to the outer surface of the shell at the ear-contacting region, the skim coating in contact with the ear tissue of the user during use.

Example 5 includes the ear-wearable device of any one of examples 1-4, wherein the ear-contacting region deviates from an ear geometry causing an interference fit between the ear-contacting region and an ear of the user. Example 6 includes the ear-wearable device of any one of examples 1-5, wherein the ear tissue includes at least a tragal wall.

Example 7 includes the ear-wearable device of any one of examples 1-6, further comprising a compliant mounting structure between the photoplethysmography sensor and the mounting void, the compliant mounting structure causing a pressure applied between the distal end of the optical transmission structure and the ear tissue to be within a predetermined pressure range during the use of the ear-wearable device. Example 8 includes the ear-wearable device of example 7, wherein the compliant mounting structure comprises a foam encapsulation around the optical transmission structure.

Example 9 includes the ear-wearable device of example 7, wherein the compliant mounting structure comprises is formed by 3-D printing a softer material at the ear-contacting region integrally with a stiffer material that is 3-D printed to form a remainder of the shell. Example 10 includes the ear-wearable device of example 7, wherein the compliant mounting structure comprises an elastomer boot that surrounds the optical transmission structure. Example 11 includes the ear-wearable device of example 10, wherein the elastomer boot further comprises: a glue pocket wing on a first side of the elastomer boot; and a snap finger on a second side opposed to the first side, and wherein the shell further comprises interface features formed contiguously with the shell extending into the internal volume of the shell, the interface features including: a snap window having a snap void through which the snap finger attaches; and a bonding surface to which the glue pocket wing is attached. Example 12 includes the ear-wearable device of example 11, wherein the interface features further comprise one or more rigid snap fingers through which one or more edges of the elastomer boot squeeze through, the one or more snap fingers retaining the elastomer boot in the mounting void.

Example 13 includes the ear-wearable device of any one of examples 7-12, further comprising a pressure sensor coupled between the shell and the photoplethysmography sensor, the pressure sensor operable to measure or estimate the pressure applied between the distal end of the optical transmission structure and the ear tissue, a signal generated by the pressure sensor coupled to a processor of the ear-wearable device. Example 14 includes the ear-wearable device of example 13, wherein the signal generated by the pressure sensor is used to detect motion artifacts and modify audio processing by the ear-wearable device based on the motion artifacts. Example 15 includes the ear-wearable device of example 13 or 14, wherein the wherein the signal generated by the pressure sensor is used to alert the user when the pressure applied between the distal end of the optical transmission structure and the ear tissue is too low.

Example 16 includes the ear-wearable device of any one of examples 1-15, further comprising a flexible conformal covering over the distal end of the photoplethysmography sensor and portions of the outer surface of the shell surrounding the distal end. Example 17 includes the ear-wearable device of example 16, wherein the flexible conformal covering comprises a fabric.

Example 18 is an ear-wearable electronic device comprising: a shell having an outer surface; a mounting void through the outer surface of the shell that exposes an internal volume of the shell, the mounting void located at an ear-contacting region of the shell; a biometric sensor assembly having a distal end exposed proximate the outer surface, the distal end of the biometric sensor conforming to the outer surface of the shell at the ear-contacting region, the distal end in contact with ear tissue of a user during use; and a compliant mounting structure between the biometric sensor and the mounting void, the compliant mounting structure causing a pressure applied between the distal end of the biometric sensor and the ear tissue to be within a predetermined pressure range during the use of the ear-wearable device.

Example 19 includes the ear-wearable device of example 18, wherein the outer surface of the shell corresponds uniquely to an ear geometry of the user of the ear-wearable device. Example 20 includes the ear-wearable device of example 18 or 19, wherein the ear-contacting region deviates from an ear geometry causing an interference fit between the ear-contacting region and a surface of the user's ear. Example 21 includes the ear-wearable device of any one of examples 18-20, wherein the ear tissue includes at least a tragal wall. Example 22 includes the ear-wearable device of any one of example 18-21, wherein the compliant mounting structure comprises a foam encapsulation around part of the biometric sensor.

Example 23 includes the ear-wearable device of any one of examples 18-21, wherein the compliant mounting structure comprises an elastomer boot that surrounds part of the biometric sensor. Example 24 includes the ear-wearable device of example 23, wherein the elastomer boot further comprises: a glue pocket wing on a first side of the elastomer boot; and a snap finger on a second side opposed to the first side, and wherein the shell further comprises interface features formed contiguously with the shell extending into the internal volume of the shell, the interface features including: a snap window having a snap void through which the snap finger attaches; and a bonding surface to which the glue pocket wing is attached. Example 25 includes the ear-wearable device of example 24, wherein the interface features further comprise one or more rigid snap fingers through which one or more edges of the elastomer boot squeeze through, the one or more rigid snap fingers retaining the elastomer boot in the mounting void.

Example 26 includes the ear-wearable device of any one of examples 18-25, further comprising a pressure sensor coupled between the shell and the biometric sensor, the pressure sensor operable to measure the pressure applied between the distal end of the biometric sensor and the ear tissue, a signal generated by the pressure sensor coupled to a processor of the ear-wearable device. Example 27 includes the ear-wearable device of example 26, wherein the signal generated by the pressure sensor is used to detect motion artifacts and modify audio processing based on the motion artifacts. Example 28 includes the ear-wearable device of examples 26 or 27, wherein the wherein the signal generated by the pressure sensor is used to alert the user when the pressure applied between the distal end of the biometric sensor and the ear tissue is too low. Example 29 includes the ear-wearable device of any one of examples 18-28, further comprising a fabric covering the distal end of the biometric sensor and portions of the outer surface of the shell surrounding the distal end.

Example 30 is a method, comprising: 3D-printing a shell of an ear-wearable device, the shell comprising: an outer surface that corresponds uniquely to an ear geometry of a user of the ear-wearable device; a mounting void through the outer surface of the shell that exposes an internal volume of the shell, the mounting void located at an ear-contacting region of the shell; and a faceplate void; moving a photoplethysmography sensor assembly through the faceplate void and towards the mounting void, the photoplethysmography sensor assembly having an optical transmission structure that fits in the mounting void; and mounting the photoplethysmography sensor into the mounting void such that a distal end of the optical transmission structure is exposed proximate the outer surface, the distal end of the optical transmission structure that conforming to the outer surface of the shell at the ear-contacting region, the distal end being in contact with ear tissue of the user during use.

Example 31 includes the method of example 30, further comprising applying a skim coating over the distal end of the optical transmission structure, the skim coating sealing the optical transmission structure and conforming to the outer surface of the shell. Example 32 includes the method of example 30 or 31, wherein an elastomer boot surrounds the distal end of the photoplethysmography sensor, the method further comprising: locating a snap finger of the elastomer boot within a snap window void that extends into the internal volume of the shell; placing an adhesive in a glue pocket wing on a side of the elastomer boot facing away from the snap finger; and rotating the elastomer boot until the photoplethysmography sensor is placed into the mounting void and the glue pocket wing contacts a bonding surface of the shell that extends into the internal volume of the shell. Example 33 includes the method of example 32, wherein placing of the photoplethysmography sensor into the mounting void causes one or more edges of the elastomer boot to squeeze through one or more rigid snap fingers of the shell that extend into the internal volume of the shell.

Example 34 is a method, comprising: receiving a signal from a pressure sensor coupled between a shell of an ear-wearable device and a biometric sensor mounted within the shell via a compliant mounting structure, the pressure sensor operable to measure a pressure applied between a distal end of the biometric sensor and ear tissue of a user of the ear-wearable device; and using the signal to detect motion artifacts and modify audio processing by the ear-wearable device based on the motion artifacts.

Example 35 is a method, comprising: receiving a signal from a pressure sensor coupled between a shell of an ear-wearable device and a biometric sensor mounted within the shell via a compliant mounting structure, the pressure sensor operable to measure a pressure applied between a distal end of the biometric sensor and ear tissue of a user of the ear-wearable device; and using the signal to alert the user when the pressure applied between the distal end of the biometric sensor and the ear tissue is too low. Example 36 includes the method of example 35, wherein the signal is further used to detect motion artifacts and modify audio processing by the ear-wearable device based on the motion artifacts.

Although reference is made herein to the accompanying set of drawings that form part of this disclosure, one of at least ordinary skill in the art will appreciate that various adaptations and modifications of the embodiments described herein are within, or do not depart from, the scope of this disclosure. For example, aspects of the embodiments described herein may be combined in a variety of ways with each other. Therefore, it is to be understood that, within the scope of the appended claims, the claimed invention may be practiced other than as explicitly described herein.

All references and publications cited herein are expressly incorporated herein by reference in their entirety into this disclosure, except to the extent they may directly contradict this disclosure. Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The recitation of numerical ranges by endpoints includes all numbers subsumed within that range (e.g. 1 to 5 includes 1, 1.5, 2, 2.75, 3, 3.80, 4, and 5) and any range within that range. Herein, the terms “up to” or “no greater than” a number (e.g., up to 50) includes the number (e.g., 50), and the term “no less than” a number (e.g., no less than 5) includes the number (e.g., 5).

The terms “coupled” or “connected” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out at least some functionality (for example, a radio chip may be operably coupled to an antenna element to provide a radio frequency electric signal for wireless communication).

Terms related to orientation, such as “top,” “bottom,” “side,” and “end,” are used to describe relative positions of components and are not meant to limit the orientation of the embodiments contemplated. For example, an embodiment described as having a “top” and “bottom” also encompasses embodiments thereof rotated in various directions unless the content clearly dictates otherwise.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like. The term “and/or” means one or all of the listed elements or a combination of at least two of the listed elements.

The phrases “at least one of,” “comprises at least one of,” and “one or more of” followed by a list refers to any one of the items in the list and any combination of two or more items in the list.

1. An ear-wearable electronic device comprising:
a shell having an outer surface;
a mounting void through the outer surface of the shell that exposes an internal volume of the shell, the mounting void located at an ear-contacting region of the shell; and
a photoplethysmography sensor assembly having an optical transmission structure mounted in the mounting void and having a distal end exposed proximate the outer surface, the distal end of the optical transmission structure conforming to the outer surface of the shell at the ear-contacting region, the distal end in contact with ear tissue of a user of the ear-wearable electronic device during use.

2. The ear-wearable device of claim 1, wherein the outer surface of the shell corresponds uniquely to an ear geometry of the user.

3. The ear-wearable device of claim 1, wherein the shell further comprises a shoulder formed contiguously with the shell extending into the internal volume of the shell, the shoulder positioned relative to the outer surface of the shell such that the distal end of the optical transmission structure is proximate to the outer surface.

4. The ear-wearable device of claim 3, further wherein the distal end of the optical transmission structure comprises a skim coating that conforms to the outer surface of the shell at the ear-contacting region, the skim coating in contact with the ear tissue of the user during use.

5. The ear-wearable device of claim 1, wherein the ear-contacting region deviates from an ear geometry causing an interference fit between the ear-contacting region and an ear of the user.

6. The ear-wearable device of claim 1, wherein the ear tissue includes at least a tragal wall.

7. The ear-wearable device of claim 1, further comprising a compliant mounting structure between the photoplethysmography sensor and the mounting void, the compliant mounting structure causing a pressure applied between the distal end of the optical transmission structure and the ear tissue to be within a predetermined pressure range during the use of the ear-wearable device.

8. The ear-wearable device of claim 7, wherein the compliant mounting structure comprises a foam encapsulation around the optical transmission structure.

9. The ear-wearable device of claim 7, wherein the compliant mounting structure comprises is formed by 3-D printing a softer material at the ear-contacting region integrally with a stiffer material that is 3-D printed to form a remainder of the shell.

10. The ear-wearable device of claim 7, wherein the compliant mounting structure comprises an elastomer boot that surrounds the optical transmission structure, wherein the elastomer boot further comprises:
a glue pocket wing on a first side of the elastomer boot; and
a snap finger on a second side opposed to the first side, and
wherein the shell further comprises interface features formed contiguously with the shell extending into the internal volume of the shell, the interface features including:
a snap window having a snap void through which the snap finger attaches; and
a bonding surface to which the glue pocket wing is attached.

11. The ear-wearable device of claim 10, wherein the interface features further comprise one or more rigid snap fingers through which one or more edges of the elastomer boot squeeze through, the one or more snap fingers retaining the elastomer boot in the mounting void.

12. The ear-wearable device of claim 7, further comprising a pressure sensor coupled between the shell and the photoplethysmography sensor, the pressure sensor operable to measure or estimate the pressure applied between the distal end of the optical transmission structure and the ear tissue, a signal generated by the pressure sensor coupled to a processor of the ear-wearable device.

13. The ear-wearable device of claim 12, wherein the signal generated by the pressure sensor is used to perform at least one of:
detecting motion artifacts and modify audio processing by the ear-wearable device based on the motion artifacts; and
alerting the user when the pressure applied between the distal end of the optical transmission structure and the ear tissue is too low.

14. The ear-wearable device of claim 1, further comprising a flexible conformal covering over the distal end of the photoplethysmography sensor and portions of the outer surface of the shell surrounding the distal end.

15. An ear-wearable electronic device comprising:
a shell having an outer surface;
a mounting void through the outer surface of the shell that exposes an internal volume of the shell, the mounting void located at an ear-contacting region of the shell;
a biometric sensor assembly having a distal end exposed proximate the outer surface, the distal end of the biometric sensor conforming to the outer surface of the shell at the ear-contacting region, the distal end in contact with ear tissue of a user during use; and
a compliant mounting structure between the biometric sensor and the mounting void, the compliant mounting structure causing a pressure applied between the distal end of the biometric sensor and the ear tissue to be within a predetermined pressure range during the use of the ear-wearable device.

16. The ear-wearable device of claim 15, wherein the outer surface of the shell corresponds uniquely to an ear geometry of the user of the ear-wearable device.

17. The ear-wearable device of claim 15, wherein the ear-contacting region deviates from an ear geometry causing an interference fit between the ear-contacting region and a surface of the user's ear.

18. The ear-wearable device of claim 15, wherein the ear tissue includes at least a tragal wall.

19. The ear-wearable device of claim 15, wherein the compliant mounting structure comprises one of a foam encapsulation around part of the biometric sensor or an elastomer boot that surrounds part of the biometric sensor.

20. The ear-wearable device of claim 15, further comprising a fabric covering the distal end of the biometric sensor and portions of the outer surface of the shell surrounding the distal end.
